(19) United States
(12) Patent Application Publication
(10) Pub. No.: US 2025/0054642 A1
(43) Pub. Date: Feb. 13, 2025

(54) EDUCATION APPLICATION FOR RECOGNIZING EVIDENCE OF MENTAL DISORDERS BY RE-USING MACHINE LEARNING DATASETS

(71) Applicant: Arizona Board of Regents on Behalf of the University of Arizona, Tucson, AZ (US)

(72) Inventor: Gondy Leroy, Tucson, AZ (US)

(21) Appl. No.: 18/797,123

(22) Filed: Aug. 7, 2024

Related U.S. Application Data
(60) Provisional application No. 63/518,072, filed on Aug. 7, 2023.

(51) Int. Cl. G16H 70/60; G16H 10/60

(52) U.S. Cl. CPC G16H 70/60; G16H 10/60

(57) ABSTRACT

An educational application that leverages gold standard data—several thousand examples of natural language sentences, extracted from electronic health records and provided by laypersons in response to surveys, describing behaviors in children indicative of autism spectrum disorder—to enable practitioners to recognize the language used by clinicians and laypersons to describe behavior indicative of autism spectrum disorder. The application provides positive examples of behaviors labeled as being indicative of one of the diagnostic criteria used to diagnose autism as well as negative examples (e.g., randomly selected from electronic health records) that are not indicative of autism spectrum disorder. Accordingly, the disclosed educational application teaches users to distinguish between relevant and non-relevant behaviors (e.g., a positive example versus a negative example), learn the accurate diagnostic criterion label (e.g., an A1 diagnostic criterion versus an A2 diagnostic criterion), and/or diagnose a case based on the combination of labels present or missing.

### CROSS-REFERENCE TO RELATED APPLICATIONS

This application claims priority to U.S. Prov. Pat. Appl. No. 63/518,072, filed Aug. 7, 2023, which is hereby incorporated by reference.

### FEDERAL FUNDING

This invention was made with government support under Grant No. MH124935 awarded by National Institutes of Health. The government has certain rights in the invention.

### SUMMARY

While the prevalence of autism spectrum disorder is rising and the age of diagnosis is not going down, there is a lack of sufficient and in-depth education of many primary care providers, pediatricians, and mental health experts. Many practitioners are insufficiently trained to diagnose autism spectrum disorder or even recognize children in need of further evaluation.

### SUMMARY

To overcome those and other drawbacks of the prior art, disclosed is an educational application that enables practitioners to recognize the language used by clinicians and laypersons to describe behavior indicative of autism spectrum disorder. The application provides positive examples, via user interface, of behaviors labeled as being indicative of one of the diagnostic criteria used to diagnose autism as well as negative examples (e.g., randomly selected from electronic health records) that are not indicative of autism spectrum disorder. Accordingly, the disclosed educational application teaches users to distinguish a relevant behavior from a non-relevant (e.g., a positive labeled example versus a negative example); to distinguish and learn the accurate diagnostic criterion label (e.g., an A1 diagnostic criterion versus an A2 diagnostic criterion); and/or to diagnose a case based on the combination of labels present or missing.

### BRIEF DESCRIPTION OF THE DRAWINGS

Aspects of exemplary embodiments may be better understood with reference to the accompanying drawings. The components in the drawings are not necessarily to scale, emphasis instead being placed upon illustrating the principles of exemplary embodiments.

FIG. 1 is a diagram of an architecture of the disclosed system according to exemplary embodiments.

FIG. 2 is a block diagram of the disclosed system according to exemplary embodiments.

FIG. 3 is a flowchart illustrating a process for providing the disclosed educational application in a criteria identification mode according to an exemplary embodiment.

FIG. 4 is a flowchart illustrating a process for providing the disclosed educational application in a single criterion mode according to an exemplary embodiment.

FIG. 5 is a flowchart illustrating a process for providing the disclosed educational application in a diagnostic mode according to an exemplary embodiment.

FIG. 6 is a flowchart illustrating a process for providing the disclosed educational application in the diagnostic mode according to another exemplary embodiment.

### DETAILED DESCRIPTION

Reference to the drawings illustrating various views of exemplary embodiments is now made. In the drawings and the description of the drawings herein, certain terminology is used for convenience only and is not to be taken as limiting the embodiments of the present invention. Furthermore, in the drawings and the description below, like numerals indicate like elements throughout.

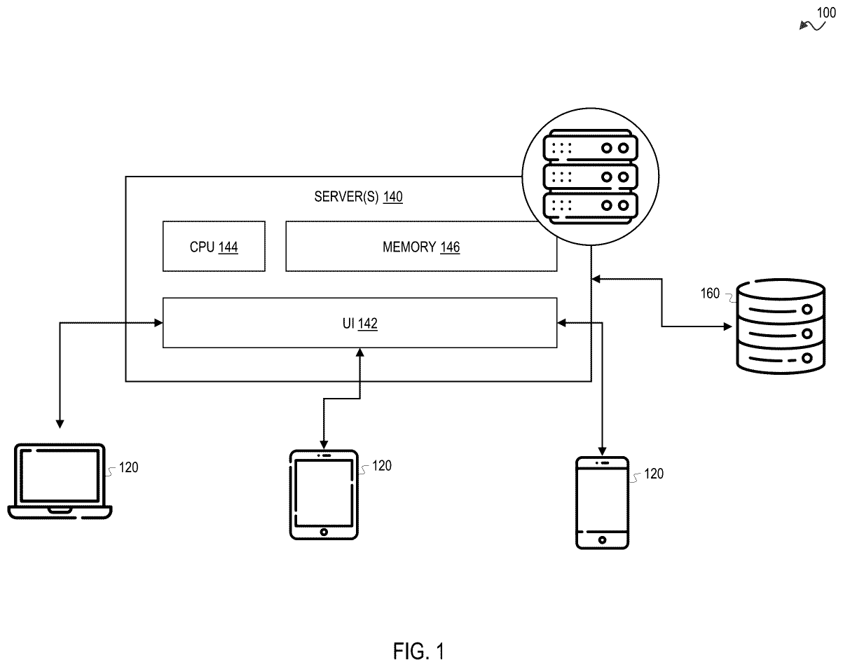

FIG. 1 is a diagram of an architecture 100 of a system for providing an educational application according to exemplary embodiments.

As shown in FIG. 1, the architecture 100 includes at least one server 140 in communication with user devices 120 via one or more computer networks 150. The server 140 stores data in non-transitory computer readable storage media 160. The server 140 may be any hardware computing device that stores instructions in memory 146 and includes at least one hardware computer processing unit 144 that executes those instructions to perform the functions described herein. The user devices 120 may include any hardware computing device having one or more hardware computer processors that perform the functions described herein. For example, the user devices 120 may include personal computers (desktop computers, notebook computers, etc.), tablet computers, smartphones, etc. The computer network(s) 150 may include any combination of wired and/or wireless communications networks, including local area networks, wide area networks (such as the internet), etc.

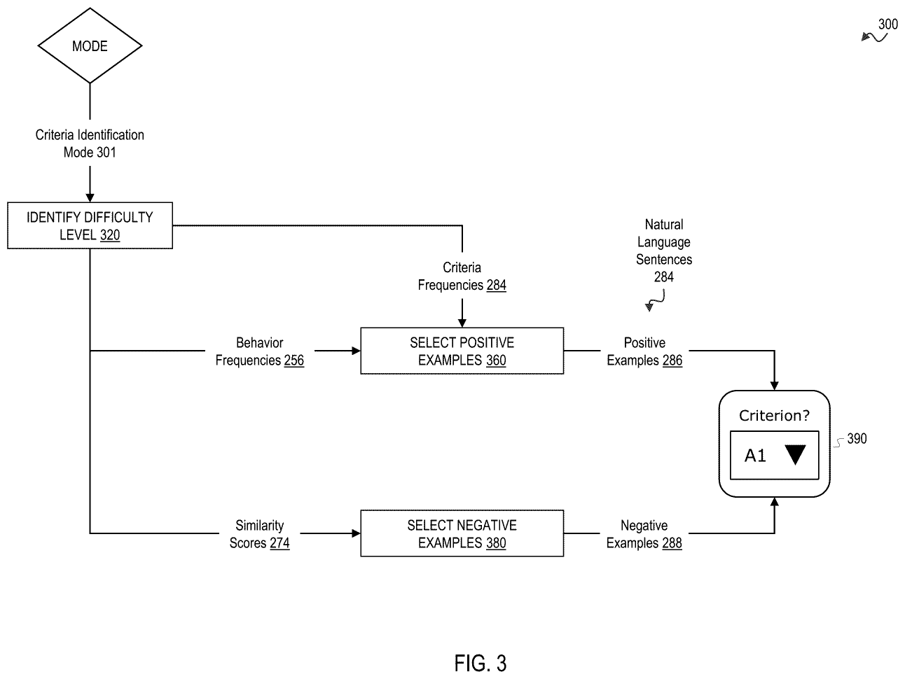

The server 140 provides a user interface 142 that enables the server 140 to provide information to and receive information from the user devices 120. The user interface 142 may be a graphical user interface (e.g., as shown in FIGS. 3-6), a command line interface, etc. In some embodiments, the user interface 142 may be realized as AI-powered avatars (e.g., synthetic parents of an autistic child discussing autistic behaviors across the spectrum) as described below.

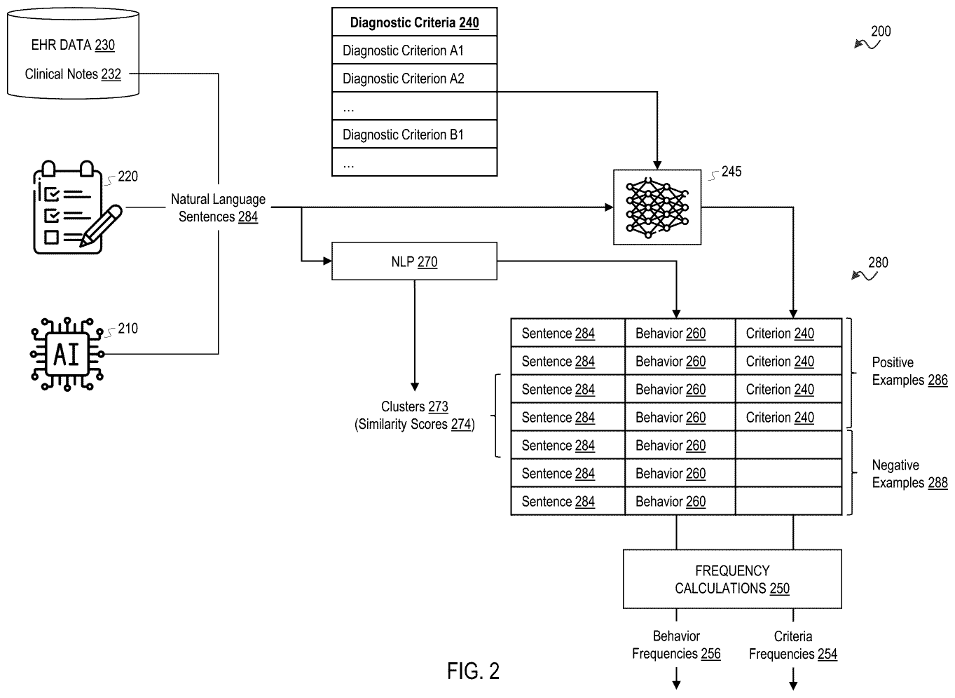

FIG. 2 is a block diagram of a system 200 for providing an educational application according to exemplary embodiments.

As shown in FIG. 2, the system 200 leverages a dataset 280 of natural language sentences 284—described in detail in U.S. patent application Ser. No. 18/797,060, which is hereby incorporated by reference—that have been reviewed by an expert clinical reviewer or labeled by a machine learning model 245 trained using reference data labeled by the expert clinical reviewer as being positive examples 286 indicative of one or more diagnostic criteria 240 used to diagnose and/or assess the severity of a mental disorder or other medical condition or negative examples 288 that are not indicative of any of the diagnostic criteria 240. For instance, in some embodiments, the system 200 leverages natural language sentences 284 labeled as indicative of the diagnostic criteria 240 used to diagnose autism spectrum disorder as shown in Table 1:

TABLE 1

| Criterion | DSM Description (extract) | EHR Example |
|---|---|---|
| A1 | Deficits in social-emotional | He appeared fairly indifferent to his peers and did |
| | reciprocity, . . . | not initiate any interactions. |
| | | Both parent and teacher rate him as having poor |
| | | pragmatic language skills. |
| A2 | Deficits in nonverbal | Does not use appropriate social communication |
| | communicative behaviors | eye contact, etc.. |
| | used for social interaction, . . . | Does not make eye contact. |
| A3 | Deficits in developing, | During free play, he played by himself with blocks |
| | maintaining, and | and miniature replications of road signs. |
| | understanding relationships, | At home, mother reports he prefers to play by |
| | . . . | himself. |
| B1 | Stereotyped or repetitive | Mother's concerns include that she seems to repeat |
| | motor movements . . . | word a lot and will say the same sentence over and |
| | | over and over again. |
| | | She repeats phrases that are said. She is prone to |
| | | engage in repetitive activities. |
| B2 | Insistence on sameness | Both at home and school, he becomes very upset if |
| | | the routine is changed. |
| | | His mother reports that he has difficulty adapting |
| | | to changing situations and that he takes longer to |
| | | recover from difficult situations than most others |
| | | his age. |
| B3 | Highly restricted, fixated | had some difficulty interrupting this pattern once it |
| | interests that are abnormal in | started and even though she clearly enjoys joint |
| | intensity or focus | play she became fixated on her simple back and |
| | | forth movement with the objects. |
| | | She plays with the same things over and over she |
| | | is described to be fixated on superheroes |
| B4 | Hyper- or hyporeactivity to | He was observed to be sensitive to unexpected |
| | sensory input | noises by covering his ears. |
| | | He demonstrates stereotypical movements, |
| | | irregularities and impairments in communication, |
| | | and unusual responses to sensory experiences. |

As shown in FIG. 2, the natural language sentences 284 may be extracted from clinical notes 232 included in electronic health records 230 (in some instances, from individuals diagnosed with autism spectrum disorder). Additionally, the system 200 may also present natural sentences 284 provided by laypersons (e.g., as part of survey data 220) using lay language to describe examples of one or more of the diagnostic criteria 240 used to diagnose autism spectrum disorder. Additionally, in some embodiments, the system 200 may present synthetic natural language sentences 284 generated by a language model 210. The synthetic natural language sentences 284 may be labeled by the machine learning model 245 as indicative of one or more diagnostic criteria 240 (or not indicative of any of the diagnostic criteria 240) or may be generated by a language model 210 prompted to generate synthetic natural language sentences 284 that are indicative of one or more diagnostic criteria 240 (or not indicative of any of the diagnostic criteria 240). Accordingly, the disclosed system 200 can be used to educate practitioners to recognize descriptions of behavior, in the language used by both clinicians and laypersons, indicative of the diagnostic criteria 240 used to diagnose autism spectrum disorder or other mental disorders.

As described in detail below, in some embodiments the system 200 also uses natural language processing 270 (e.g., a machine learning classification model) and other algorithms (e.g., frequency calculations 250) to present learning at various difficulty levels.

For instance, the because the disclosed system 200 includes a large dataset 280 of real examples (excluding the synthetic natural language sentences 284 generated by a language model 210), the system 200 may calculate the relative frequency of each individual diagnostic criterion 240 (criteria frequencies 254). In the autism spectrum disorder example, for instance, the most frequent diagnostic criterion 240 may be A1 (e.g., identified in over 25% of all electronic health records 230 of individuals diagnosed with autism spectrum disorder) while the least frequent diagnostic criterion may be B3 (e.g., identified in less than 5% of all electronic health records 230 of individuals diagnosed with autism spectrum disorder) as shown in Table 2:

TABLE 2

| Diagnostic Criterion 240 | Count | Diagnostic Frequency 254 |
|---|---|---|
| A1 | 1,524 | 25.11% |
| . . . | . . . | . . . |
| B3 | 277 | 4.56% |
| Total | 6,069 | 100.00% |

Having calculated the criteria frequencies 254 of each diagnostic criterion 240, the system 200 may present users at lower difficulty levels with examples of diagnostic criterion 240 that are more common while presenting users who have progressed to increasingly higher difficulty levels with examples of diagnostic criterion 240 that are increasingly less common.

Additionally, each diagnostic criterion 240 may include a number of distinct behaviors 260 with some—but not all—examples of those behaviors 260 being indictive of that diagnostic criterion 240. Accordingly, in some embodiments, the system 200 includes natural language processing 270 that classifies each natural language sentence 284 as indicative of a particular behavior 260. Again, because the disclosed system 200 includes a large dataset 280 of real examples, the system 200 may calculate the relative frequency of each identified behavior 260 (behavior frequencies 256). The behavior frequencies 256 may be calculated using the entire dataset 280 or a representative sample of the dataset 280. Because synthetically created examples (i.e., generated by the language model 210) may introduce incorrect frequencies, however, those behavior frequencies 256 may be calculated using a dataset that excludes those synthetically created examples. Meanwhile, having the behavior frequencies 256 of each identified behavior 260, the system 200 may present users at lower difficulty levels with examples of behaviors 260 that are more common while presenting users who have progressed to increasingly higher difficulty levels with examples of behaviors 260 that are increasingly less common.

Additionally, the system 200 may cluster new, unlabeled natural language sentences 284 into clusters 273 of natural language sentences 284 that are indicative similar behaviors 260, for example by calculating similarity scores 274 indicative of the similarity between the identified behaviors 260 for each pair of natural language sentences 284. Notably, each cluster 273 of natural language sentences 284 indicative of similar behaviors 260 may include both positive examples 286 of one or more diagnostic criteria 240 as well as negative examples 288 that contain some information relevant to one or more of the diagnostic criteria 240 but were nevertheless labeled by the machine learning model 245 or the expert clinical reviewer as not being indicative of any of the diagnostic criteria 240 (or were generated by a language model 210 as synthetic natural language sentences 284 that are not indicative of one or more diagnostic criteria 240.

In those embodiments, having calculated similarity scores 274 for each of the natural language sentences 284, the system 200 may present users at lower difficulty levels with negative examples 288 that have low similarity scores 274 when compared to positive examples 286 of one or more of the diagnostic criteria 240 while presenting users at increasingly higher difficulty levels with negative examples 288 that have increasingly higher similarity scores 274 with positive examples 286 (e.g., negative examples 288 with high similarity scores 274 when compared to positive examples 286 of diagnostic criterion A1 that were nevertheless labeled as a negative example 288 of diagnostic criterion A1 during the clinical review 245).

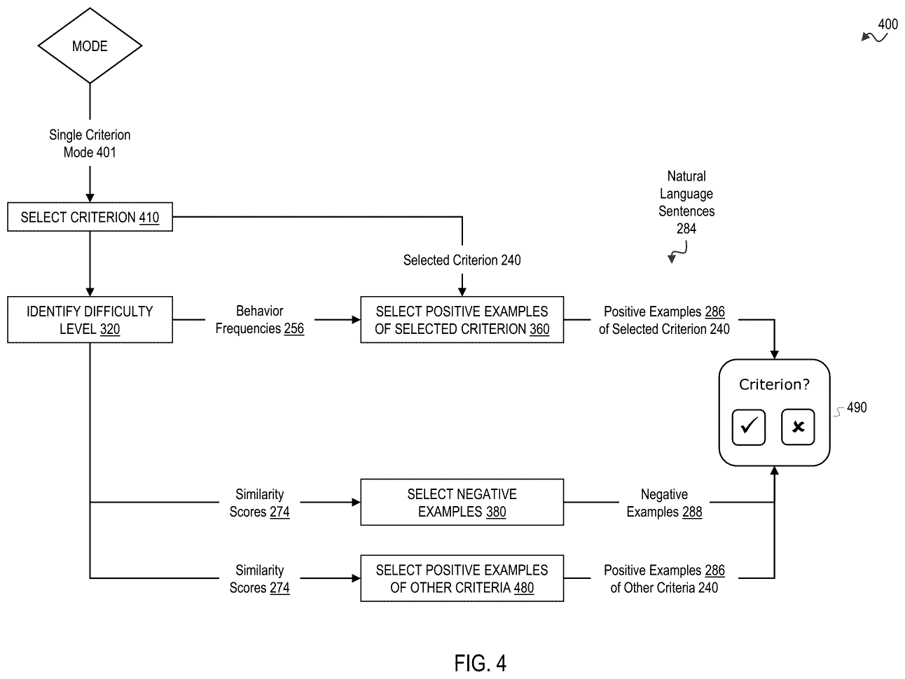

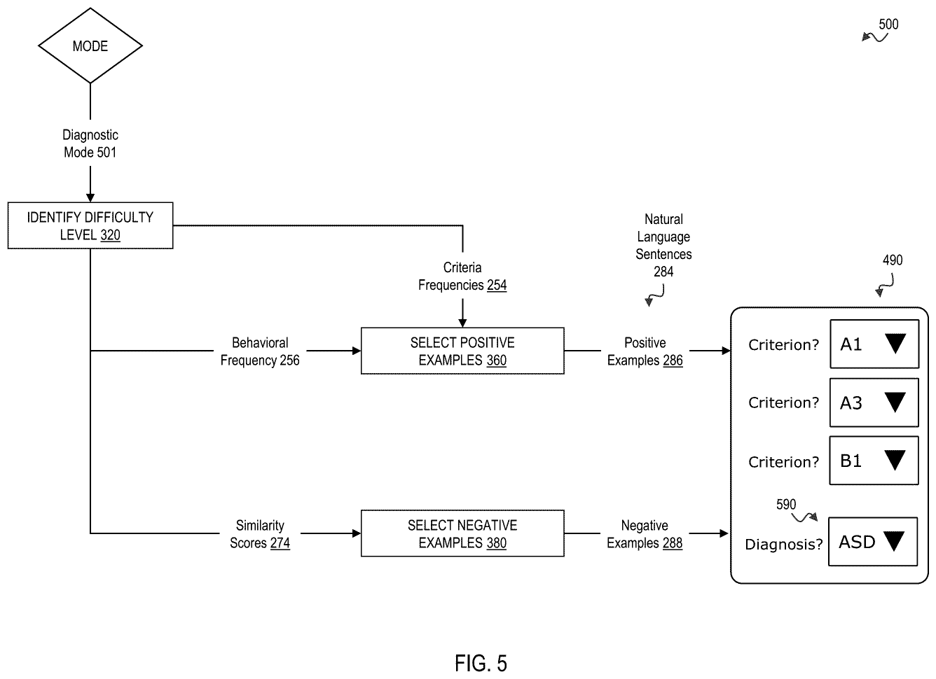

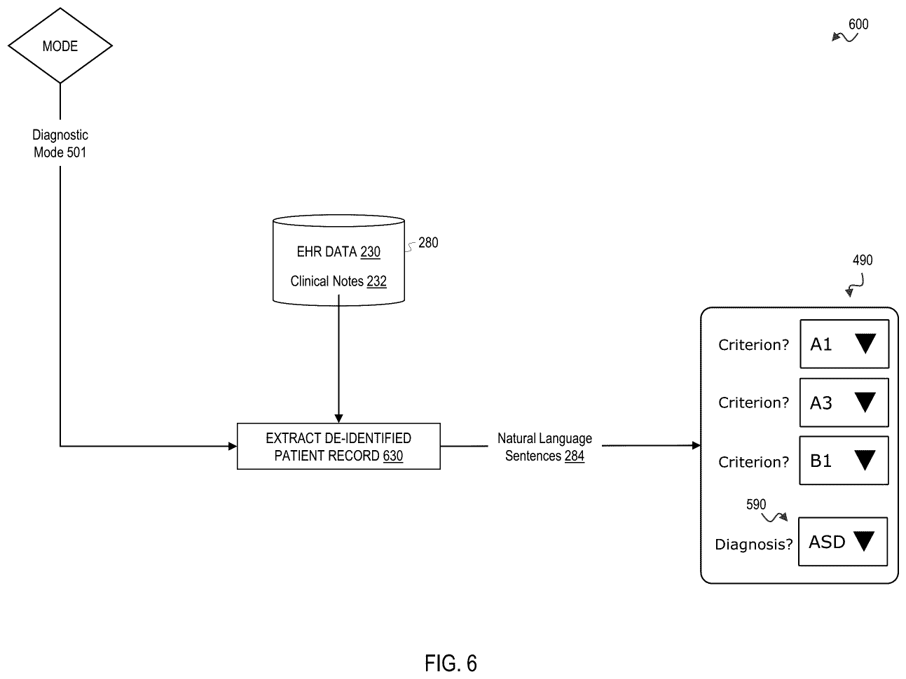

The overall goal of the educational application is to provide examples (positive examples 286 and negative examples 288) of behaviors 260 (e.g., of children diagnosed with autism spectrum disorder) and to teach learners:
To distinguish a relevant behavior 260 from a non-relevant behavior 260, e.g., a positive labeled example 286 versus a negative example 288, for example as described below with reference to FIGS. 3-4;
To distinguish and learn the accurate diagnostic criterion label 240, e.g., diagnostic criterion A1 versus diagnostic criterion A2, for example as described below with reference to FIGS. 3-4; and
To diagnose a case based on the combination of labels present or missing, for example as described below with reference to FIGS. 5-6.

FIG. 3 is a flowchart illustrating a process 300 for providing the disclosed educational application in a criteria identification mode 301 according to an exemplary embodiment.

As shown in FIG. 3, natural language sentences 284 selected from the dataset 280 are presented to the user along with functionality 390 for the user to indicate whether the presented natural language sentence 284 is indicative of one or more diagnostic criteria 240. The educational application then determines whether the user response is consistent with the label provided by the expert during the clinical review process 245 described above.

In the example process 300 of FIG. 3, positive examples 286 are selected in step 360 and negative examples 288 are selected in step 380. In some embodiments, a difficulty level is identified in step 320. In those embodiments, the positive examples 286 and the negative examples 288 may be selected in accordance with the identified difficulty level. For example, the system 200 may select positive examples 286 of diagnostic criterion 240 that are more common (having higher criteria frequencies 284) for users at lower difficulty levels while selecting positive examples 286 of diagnostic criterion 240 that are increasingly less common for users who have progressed to increasingly higher difficulty levels. Similarly, the system 200 may select positive examples 286 of behaviors 260 that are more common (having higher behavior frequencies 256) for users at lower difficulty levels while selecting positive examples 286 of behaviors 260 that are increasingly less common (with increasingly lower behavior frequencies 256) for users who have progressed to increasingly higher difficulty levels. Additionally, the system 200 may select negative examples 288 with lower similarity scores 274 for users at lower difficulty levels while presenting users at increasingly higher difficulty levels with negative examples 288 that have increasingly higher similarity scores 274.

FIG. 4 is a flowchart illustrating a process 400 for providing the disclosed educational application in a single criterion mode 401 according to an exemplary embodiment.

As shown in FIG. 4, a diagnostic criterion 240 is selected in step 410 and natural language sentences 284 selected from the dataset 280 are presented to the user along with functionality 490 for the user to indicate whether the presented natural language sentence 284 is indicative of the selected diagnostic criterion 240. The educational application then determines whether the user response is consistent with the label provided by the expert during the clinical review process 245 described above.

As described above with reference to FIG. 3, positive examples 286 are selected in step 360 and negative examples 288 are selected in step 380. Additionally, in the single criterion mode 401, the user is also presented with positive examples 286 of other diagnostic criteria 240 (i.e., the diagnostic criteria 240 not selected in step 410) to learn to distinguish positive examples 286 of the diagnostic criterion 240 selected in step 410 and the other diagnostic criteria 240.

Again, in some embodiments, a difficulty level is identified in step 320 and the positive examples 286 and/or the negative examples 288 may be selected in accordance with the identified difficulty level. Again, the system 200 may select positive examples 286 of behaviors 260 that are more common (having higher behavior frequencies 256) for users at lower difficulty levels while selecting positive examples 286 of behaviors 260 that are increasingly less common (with increasingly lower behavior frequencies 256) for users who have progressed to increasingly higher difficulty levels. The system 200 may select negative examples 288 with lower similarity scores 274 for users at lower difficulty levels while presenting users at increasingly higher difficulty levels with negative examples 288 that have increasingly higher similarity scores 274.

Additionally, in step 480 of the single criterion mode 401, the system 200 may select positive examples 486 of the other diagnostic criteria 240 having lower similarity scores 274 (when compared to positive examples 286 of the selected criterion 240) for users at lower difficulty levels while presenting users at increasingly higher difficulty levels with positive examples 288 of the other diagnostic criteria 240 that have increasingly higher similarity scores 274 (compared to positive examples 286 of the selected criterion 240).

FIG. 5 is a flowchart illustrating a process 500 for providing the disclosed educational application in a diagnostic mode 501 according to an exemplary embodiment.

As shown in FIG. 5, multiple natural language sentences 284 selected from the dataset 280 are presented to the user along with functionality 490 for the user to indicate whether the presented natural language sentence 284 is indicative of one or more diagnostic criteria 240 and functionality 590 for the user to indicate whether a hypothetical patient described using all of those natural language sentences 284 provided should be diagnosed with autism spectrum disorder under established medical guidelines (e.g., having at least three A criteria 240 and examples of at least two B criteria 240 as indicated by the DSM5). The educational application then determines whether the user responses are consistent with the labels provided by the expert during the clinical review process 245 described above.

In the example process 500 of FIG. 5, the positive examples 286 are selected in step 360 and negative examples 288 are selected in step 380 in accordance with a difficulty level identified in step 320. (As described above, for example, the system 200 may select positive examples 286 of behaviors 260 and diagnostic criterion 240 that are more or less common—and negative examples 288 that are more or less similar—depending on the identified difficulty level.)

FIG. 6 is a flowchart illustrating a process 600 for providing the disclosed educational application in the diagnostic mode 501 according to another exemplary embodiment.

In the example process 600 of FIG. 6, the natural language sentences 284 presented to the user are selected by extracting the natural language sentences 284 included the clinical notes 232 of a single, de-identified electronic health record 230. In those embodiments, the natural language sentences 284 are presented in their original form and the educational application provides functionality 490 for the user to indicate whether each presented natural language sentence 284 is indicative of one or more diagnostic criteria 240 and functionality 590 for the user to indicate whether the patient described in the electronic health record 230 should be diagnosed with autism spectrum disorder under established medical guidelines. The educational application then determines whether the user responses are consistent with the labels provided by the expert during the clinical review process 245 described above.

As briefly mentioned above, the user interface 142 may be realized as AI-power avatars. For example, the system 200 use the dataset 280 to dynamically generate synthetic parents of autistic children that discuss autistic behaviors across the spectrum. For instance, the dataset 280 may be pre-processed to generate cases that present different complexity and include behaviors 260 across the spectrum (e.g., repetitive motor movements vs. atypical social response), as well as non-autistic behaviors (e.g., shyness) that should be part of the training. Case details may be used to instantiate avatars that play a role and share the relevant behaviors 260 when appropriately asked by the clinician.

In those embodiments, pre-processing of the dataset 280 to create realistic and varied cases may include stratification by the age and gender of the child first, then identifying clusters 273 (e.g., using word embeddings to identify scores 274 of semantic similarities between words and phrases) of similar behaviors 260 within each diagnostic criterion 240. A subset of examples may be labeled by clinicians. Clinician evaluations may be correlated to the behavior frequencies 256 of each behavior 260 to create a frequency-based indication of representative ASD features.

While the disclosed system is described above in reference to autism spectrum disorder, one of ordinary skill in the art would recognize that the disclosed system can be configured to train individuals to recognize descriptions of behaviors indicative of other diagnostic criteria used to diagnose other mental disorders. While preferred embodiments have been described above, those skilled in the art who have reviewed the present disclosure will readily appreciate that other embodiments can be realized within the scope of the invention. Accordingly, the present invention should be construed as limited only by any appended claims.

What is claimed is:

1. A method, comprising:
storing sentences labeled by an expert clinical reviewer or machine learning model as being indicative or not indicative of one or more of the diagnostic criteria used to diagnose a mental disorder; and
providing a user interface that:
presents at least one of the sentences;
provides functionality for the user to indicate whether the presented sentence is indicative of one of the one or more of the diagnostic criteria used to diagnose a mental disorder; and
outputs an indication of whether the user correctly indicated whether the presented sentence is indicative of one of the one or more of the diagnostic criteria.

2. The method of claim 1, wherein at least some of the sentences are extracted from clinical notes of electronic health records of individuals diagnosed with the mental disorder labeled as being indicative of at least one of the diagnostic criteria used to diagnose the mental disorder.

3. The method of claim 2, further comprising:
calculating the frequency of each diagnostic criterion identified in the electronic health records.

4. The method of claim 3, further comprising:
at each of a plurality of increasingly higher difficulty levels, selecting sentences indicative of diagnostic criterion that are increasingly less frequent.

5. The method of claim 4, further comprising:
using natural language processing or a machine learning model to classify each sentence as describing a behavior; and
calculating the frequency of each identified behavior in the electronic health records.

6. The method of claim 3, further comprising:
at each of a plurality of increasingly higher difficulty levels, selecting sentences indicative of behaviors that are increasingly less frequent.

7. The method of claim 6, further comprising:
calculating a similar score for each pair of sentences; and
at each of a plurality of increasingly higher difficulty levels, selecting negative examples having increasingly higher similarity scores with respect to positive examples of one or more of the diagnostic criteria.

8. The method of claim 1, wherein the user interface is further configured to:
provide functionality for the user to indicate which of the diagnostic criteria the presented sentence is indicative of; and
output an indication of whether the user correctly indicated which of the diagnostic criteria the presented sentence is indicative of.

9. The method of claim 1, wherein at least some of the sentences are extracted from survey responses provided by laypersons and labeled by the expert clinical reviewer or machine learning model as being indicative or not indicative of one or more of the diagnostic criteria used to diagnose the mental disorder.

10. The method of claim 1, wherein at least some of the sentences are generated by a language model in response to a prompt asking for examples indicative or not indicative of one or more of the diagnostic criteria used to diagnose the mental disorder.

11. A system, comprising:
non-transitory computer readable storage media that stores sentences labeled by an expert clinical reviewer as being indicative or not indicative of one or more of the diagnostic criteria used to diagnose a mental disorder; and
at least one hardware computer processor that provides a user interface that:
presents one of the sentences;
provides functionality for the user to indicate whether the presented sentence is indicative of one of the one or more of the diagnostic criteria used to diagnose a mental disorder; and
outputs an indication of whether the user correctly indicated whether the presented sentence is indicative of one of the one or more of the diagnostic criteria.

12. The system of claim 11, wherein at least some of the sentences are extracted from clinical notes of electronic health records of individuals diagnosed with the mental disorder labeled as being indicative of at least one of the diagnostic criteria used to diagnose the mental disorder.

13. The system of claim 12, wherein the at least one hardware computer processor is further configured to:
calculate the frequency of each diagnostic criterion identified in the electronic health records.

14. The system of claim 13, wherein, at each of a plurality of increasingly higher difficulty levels, the at least one hardware computer processor is further configured to select sentences indicative of diagnostic criterion that are increasingly less frequent.

15. The system of claim 14, wherein the at least one hardware computer processor is further configured to:
classify each sentence, using natural language processing or a machine learning model, as describing a behavior; and
calculate the frequency of each identified behavior in the electronic health records.

16. The system of claim 13, wherein, at each of a plurality of increasingly higher difficulty levels, the at least one hardware computer processor is further configured to select sentences indicative of behaviors that are increasingly less frequent.

17. The system of claim 16, wherein the at least one hardware computer processor is further configured to:
calculate a similar score for each pair of sentences; and
at each of a plurality of increasingly higher difficulty levels, select negative examples having increasingly higher similarity scores with respect to positive examples of one or more of the diagnostic criteria.

18. The system of claim 11, wherein the user interface is further configured to:
provide functionality for the user to indicate which of the diagnostic criteria the presented sentence is indicative of; and
output an indication of whether the user correctly indicated which of the diagnostic criteria the presented sentence is indicative of.

19. The system of claim 11, wherein at least some of the sentences are extracted from survey responses provided by laypersons and labeled by the expert clinical reviewer or machine learning model as being indicative or not indicative of one or more of the diagnostic criteria used to diagnose the mental disorder.

20. The system of claim 11, wherein at least some of the sentences are generated by a language model in response to a prompt asking for examples indicative or not indicative of one or more of the diagnostic criteria used to diagnose the mental disorder.
